(19) United States
(12) Patent Application Publication
(10) Pub. No.: US 2021/0128446 A1
(43) Pub. Date: May 6, 2021

(54) COSMETIC COMPOSITION COMPRISING BIODEGRADABLE POLYMERS

(71) Applicant: L'ORÉAL, Paris (FR)

(72) Inventors: I-Fan HSIEH, Scotch Plains, NJ (US); Hy Si BUI, Piscataway, NJ (US); Mariko HASEBE, West New York, NJ (US); Sophia HUA, Tomball, TX (US)

(73) Assignee: L'ORÉAL, Paris (FR)

(21) Appl. No.: 16/669,676

(22) Filed: Oct. 31, 2019

(51) Int. Cl. A61K 8/81; A61K 8/34; A61Q 1/02

(52) U.S. Cl. CPC A61K 8/8182; A61Q 1/02; A61K 8/345

(57) ABSTRACT

Disclosed is a cosmetic composition and methods of use, where the composition includes humectant and a copolymer of vinylpyrrolidone and a carboxylic acid monomer in water where (i) the copolymer is present in an amount that is at least about 1% by weight of the total composition, (ii) the humectant is present in an amount that is less than about 10% by weight of the total composition, and (iii) the pH of the water is less than about 5, the copolymer is less than about 10% neutralized, or a combination thereof.

### FIELD OF THE INVENTION

The present invention relates to cosmetic compositions, and specifically to aqueous cosmetic compositions utilizing a humectant and a copolymer of vinylpyrrolidone and a carboxylic acid monomer in water, where the humectant is present in an amount that is less than about 10% by weight of the total composition, where the pH of the water is less than about 5 and/or the copolymer is less than about 10% neutralized.

### BACKGROUND

Cosmetic products such as facial primers and foundation are expected to have good wear and transfer resistance properties, especially resistance to migration and transfer when the product gets wet, such as when a user begins to sweat. Rheology and film formation is critical, and the cosmetics industry spends significant effort balancing the competing performance characteristics desired in a particular product, while also maintaining pleasing application characteristics. Therefore, a cosmetic product with improved sweat resistance while maintaining a desirable feel during application is useful and needed.

### BRIEF SUMMARY

A first aspect of the present disclosure is cosmetic composition, such as an aqueous leave-on composition, that includes a humectant and a copolymer of vinylpyrrolidone and a carboxylic acid monomer, such as acrylic acid, in water. The copolymer is present in an amount that is at least about 1% by weight of the total composition, the humectant is present in an amount that is less than about 10% by weight of the total composition, and the pH of the water is less than about 5, the copolymer is less than about 10% neutralized, or a combination thereof. Optionally, the pH of the water is less than about 5 and the copolymer is less than about 10% neutralized. Optionally, the pH of the water is less than 4. Optionally, the copolymer is less than 5% neutralized. Optionally, the copolymer is present in an amount of between 1% and 10% by weight of the total composition. Optionally, the humectant is present in an amount of between 0.1% and 6% by weight of the total composition. Optionally, the humectant may be a diol or triol, such as butylene glycol.

Optionally, the composition is a solution, a mixture, or an may comprise at least two immiscible components prior to application. In some embodiments, it is a water-in-oil emulsion or an oil-in-water emulsion. In some embodiments, it is a gel.

Optionally, the composition may include other components, such as a colorant, where the colorant may be, e.g., an inorganic pigment. Optionally, the composition includes a skin active agent, skin adjuvant, or UV filter. Optionally, the composition includes a mattifying agent selected from the group consisting of a talc, silica, silicone elastomer, polyamide, wax, and combinations thereof.

Optionally, the copolymer is the only rheology modifying agent or film former in the composition.

A second aspect of the present disclosure is a method for providing a long-lasting composition to the skin. The method includes first applying to a person's skin a composition comprising a copolymer of vinylpyrrolidone and a carboxylic acid monomer in water and a humectant, and then allowing the person to sweat, such that the sweat from the person increases the composition's overall resistance to deformation.

A third aspect of the present disclosure is a method for providing a composition with an improved wear resistance property, comprising the steps of first providing a copolymer of vinylpyrrolidone and a carboxylic acid monomer in water, then adding a neutralizing agent at an amount sufficient to neutralize less than 10% of the copolymer and/or adjust the pH of the water is less than about 5, then providing a humectant. The copolymer should be present in an amount that is at least about 1% by weight of the total composition, and the humectant should be present in an amount that is at least about 10% by weight of the total composition. Optionally, the neutralizing agent comprises amines.

### BRIEF DESCRIPTION OF THE DRAWINGS

FIG. 1 is a graph illustrating the complex shear modulus of a base composition at different levels of neutralization, where the copolymer of vinylpyrrolidone and a carboxylic acid monomer was kept at a constant amount of 2.5% by weight.

FIG. 2 is a graph illustrating the complex shear modulus of a base composition without humectant, after the addition of varying levels of either sweat (solid circles, solid line) or additional water (open boxes, dashed lines).

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the terms “between [two numbers]” is intended to include those two numbers. For example, “x is between 1 and 2” is intended to cover 1≤x≤2.

As used herein, the terms “include”, “includes” and “including” are meant to be non-limiting.

A first aspect of the present disclosure is drawn to a cosmetic composition, such as an aqueous leave-on composition. The composition includes a humectant and a copolymer of vinylpyrrolidone and a carboxylic acid monomer, where the pH of the water is less than about 5 and/or the copolymer is less than about 10% neutralized. As one of skill will recognize, “the pH of the water” refers to the measured pH of the solution if the composition is a solution, the measured pH of the aqueous phase if the composition is an emulsion, etc. In some embodiments, the pH of the water is less than about 5 and the copolymer is less than about 10% neutralized. In some embodiments, the pH of the water is less than about 4.5. In some embodiments, the pH of the water is less than about 4 (such as between 3 and 4). In some embodiments, the copolymer is less than 5% neutralized.

The composition may take various forms, including a solution, a mixture, or an emulsion, and as such may comprise at least two immiscible components prior to application. In some embodiments, the composition is a water-in-oil emulsion or an oil-in-water emulsion. In some embodiments, it is a gel.

Humectant

The humectant is utilized in the composition in an amount of less than about 10% by weight, such as less than about 8% by weight, or between 0.1% and 6% by weight, relative to the total weight of the composition.

Polyhydric alcohols, preferably those of C₂-C_{8,} more preferably a C₃-C_{6,} preferably, for example, glycerol, propylene glycol, 1,3-butylene glycol, pentylene glycol, hexylene glycol, dipropylene glycols, diethylene glycol, and diglycerol, and mixtures thereof, glycerol and their derivatives are known as humectants or wetting agents.

Non-limiting examples of other humectants which may be utilized include glycerol and its derivatives, urea and its derivatives, especially Hydrovance marketed by National Starch, lactic acid, hyaluronic acid, AHA, BHA, sodium pidolate, xylitol, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NutraLipids, a C-glycoside derivatives, in particular the C-13-D-xylopyranoside-2-hydroxypropane in the form of a solution at 30% by weight of active material in a water/propylene glycol mixture (60/40 wt %) as the product produced by the company Chimex under the trade name “Mexoryl SBB”, a rose hip oil marketed by Nestle, a micro-algae extract Prophyridium cruentum enriched with zinc, marketed under the name by Vincience Algualane Zinc spheres of collagen and chondroitin sulfate of marine origin (Atelocollagen) sold by the company Engelhard Lyon under the name Marine Filling Spheres, hyaluronic acid spheres such as those marketed by Engelhard Lyon, and arginine.

Preferably, the humectant may be a diol or triol, such as butylene glycol.

In the disclosed compositions, the humectant typically increases hydration and playtime properties (e.g., redeposition, retouching, etc.) of the composition.

Copolymer of vinylpyrrolidone and a carboxylic acid monomer

The copolymer of vinylpyrrolidone and a carboxylic acid monomer is utilized in the composition in an amount of greater than about 1% by weight, such as between about 1% and about 10% by weight, relative to the total weight of the composition. The copolymer is generally present in water.

Suitable carboxylic acid monomers include (meth)acrylic acid, crotonic acid, itaconic acid, maleic acid and mixtures thereof, preferably acrylic acid.

In particular, a vinylpyrrolidone/acrylic acid copolymer is preferred, such as that sold as Ultrathix™ P-100 polymer Ashland Inc. (INCI name Acrylic Acid/VP Crosspolymer).

The copolymer may be neutralized with a suitable neutralizing agent. In preferred embodiments, the copolymer is neutralized in water or in an aqueous solution with a neutralizing agent before the polymer is added into the disclosed cosmetic composition. In other preferred embodiments, the at least one acrylic polymer of the present invention is neutralized with a neutralizing agent at the time of addition of the polymer into the disclosed cosmetic composition.

The neutralizing agent is employed in an amount sufficient to neutralize some of the carboxylic acid monomer. After neutralization, the copolymer should be partially neutralized, and preferably less than about 10% neutralized. One indication of neutralization is the clarity of the solution.

Non-limiting examples of neutralizing agents may be selected from alkali metal carbonates, alkali metal phosphates, organic amines, hydroxide base compounds, and mixtures thereof, particularly from organic amines, alkali metal hydroxides, alkali earth metal hydroxides, and mixtures thereof.

Organic amines may be selected from amino-2-methyl-1-propanol (or aminomethyl propanol), ethylamines, ethyleneamines, alkanolamines, cyclic amines and other cyclic compounds, saturated or unsaturated, having one or more nitrogen atoms within the ring, and mixtures thereof. Organic amines may also be chosen from alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Among the compounds of the alkanolamine type that may be mentioned include but not limited to: monoethanolamine (also known as monoethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino)methane.

Other examples include but are not limited to 1,3-diaminopropane, 1,3-diamino-2-propanol, spermine, and spermidine.

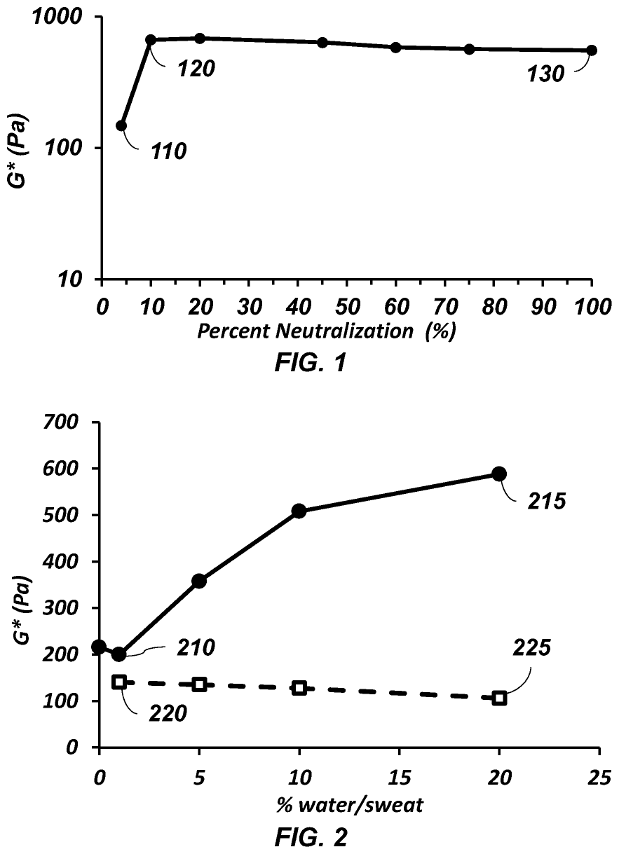

Surprisingly, with reference to FIG. 1, it was found that the rheology of the disclosed compositions, and the complex shear modulus (G*) in particular, depend greatly on how much neutralization has occurred. The complex shear modulus correlates with a material's resistance to deformation. At neutralization of less than about 10%, the complex shear modulus is highly sensitive to neutralization. In FIG. 1, the complex shear modulus at 4% neutralization (110) is approximately 148 Pa, while at 10% neutralization (120), the complex shear modulus is approximately 665 Pa. Above about 10% neutralization (120), however, viscoelastic properties among various neutralizations do not change significantly, with the complex shear modulus at 100% neutralization (130) being approximately 554 Pa. The rheological characteristics were measured using a MCR 502 from ANTON PAAR rotational rheometer, equipped with an air-cooled Peltier plate to regulate the temperature at 25° C. A cone and plate geometry 50 mm/angle 1° (sandblasted stainless steel at 5μm) was used with anti-evaporation device to avoid evaporation during measurements. While not shown, a similar graph can be seen when looking at the impact of pH on rheology. As the pH increases from a very low pH to about 5, and more particularly to about 4.5, and still more particularly to about 4, the complex shear modulus increases with pH. Above about 5, more particularly above about 4.5, and still more particularly above about 4, the complex shear modulus no longer increases with pH, with no significant changes in complex shear modulus as pH increases to about 7.

Optionally, the composition may include other components, such as a colorant, a preservative, a skin active agent, a skin adjuvant, a UV filter, a mattifying agent, or an additional rheology modifying agent or additional film former.

Colorant

The cosmetic compositions may contain at least one cosmetically acceptable colorant such as a pigment or dyestuff. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface treated within the scope of the present invention but are not limited to treatments such as silicones, perfluorinated compounds, lecithin, and amino acids.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

The precise amount and type of colorant employed in embodiments of the disclosed compositions will depend on the color, intensity and use of the cosmetic composition and, as a result, will be determined by those skilled in the art of cosmetic formulation. However, one preferred amount of colorant for use in the present invention is from about 0.5% to about 7.5%, based on the weight of the composition.

Preservatives

One or more preservatives may be included in the compositions described herein for treating hair. Suitable preservatives include, but are not limited to, glycerin containing compounds (e.g., glycerin and/or ethylhexylglycerin and/or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the composition. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol).

The preservative is optionally included in an amount ranging from about 0.01 wt. % to about 5 wt. %, about 0.05% to about 4 wt. %, or about 0.06 wt. % to about 3 wt. %, based on the total weight of the composition.

Skin Active Agents

The cosmetic compositions may contain at least one skin-active agents, which may include an anti-aging agent, a depigmenting agent, an anti-wrinkle agent, or an agent that treats oily skin. Non-limiting examples of active skin-agents include adenosine, 2-[4-(2-hydroxyethyl)piperazin-1-yl]ethanesulfonic acid (HEPES), lanolin, citric acid, malic acid, tartaric acid, salicylic acid, vitamin C, a vitamin, a retinoid, retinal, retinoic acid, a carotenoid, an amino acid, a protein, an enzyme, and a coenzyme.

Depigmenting agents include vitamin C and its derivatives and especially vitamin CG, CP and 3-O ethyl vitamin C, alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, ceramides and their counterparts, derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include vitamin C and its derivatives and especially vitamin CG, CP and 3-0 ethyl vitamin C, alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

The term “anti-wrinkle active” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Exemplary anti-wrinkle actives may be chosen from: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof.

Examples of such compounds are: adenosine and its derivatives and retinol and its derivatives such as retinol palmitate, ascorbic acid and its derivatives such as magnesium ascorbyl phosphate and ascorbyl glucoside; tocopherol and derivatives thereof such as tocopheryl acetate, nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof.

As adenosine derivatives include especially non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside.

Other derivatives include adenosine receptor agonists such as adenosine phenylisopropyl (“PIA”), 1-methylisoguanosine, N6-cyclohexyl adenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

In some instances, the compositions of the instant disclosure comprise a skin-active ingredient that addresses oily skin. These actives can be sebo-regulating or antiseborrhoeic agents capable of regulating the activity of sebaceous glands. These include: retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppicthe mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech;—extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of ‘meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaj a saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C₁₂-C₁₃) sold under the name COSMACOL® ECI by Sasol; trialkyl citrate (C₁₄-C₁₅) sold under the name COSMACOL® ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

Skin Adjuvants

There are also several optional skin adjuvants that may be included. Examples include pH adjusters, conditioning agents, and chelating agents. Non-limiting examples of pH adjusters include aminomethyl propanol, aminomethylpropane diol, triethanolamine, triethylamine, citric acid, sodium hydroxide, acetic acid, potassium hydroxide, lactic acid, and combinations thereof. Non-limiting examples of conditioning agents include cyclomethicone, petrolatum, dimethicone, dimethiconol, silicone, such as cyclopentasiloxane and diisostearoyl trimethylolpropane siloxy silicate, sodium hyaluronate, isopropyl palmitate, soybean oil, linoleic acid, PPG-12/saturated methylene diphenyldiisocyanate copolymer, urea, amodimethicone, trideceth-12, cekimonium chloride, diphenyl dimethicone, tocopherol, quaternary amines, and combinations thereof. Non-limiting examples of chelating agents include ethylenediaminetetraacetic acid (EDTA), tetrasodium etidronate, tetrasodium pyrophosphate, pentasodium ethylenediamine tetramethylene phosphonate, sodium staminate and combinations thereof.

UT 7 Filters

The compositions may also optionally comprise UV filters. The UV filter may be one or more organic UV filters and/or one or more inorganic UV filters. Non-limiting examples of inorganic UV filters includes metal oxides (titanium dioxide, zinc oxide, etc.) and mixtures thereof. Non-limiting examples of inorganic UV filters include anthranilic compounds, dibenzoylmethane compounds, cinnamic compounds, salicylic compounds, camphor compounds, benzophenone compounds, β,β-diphenylacrylate compounds, triazine compounds, benzotriazole compounds, benzalmalonate compounds, benzimidazole compounds, imidazoline compounds, bis-benzoazolyl compounds, p-aminobenzoic acid (PABA) compounds, methylenebis(hydroxyphenylbenzotriazole) compounds, benzoxazole compounds, screening polymers and screening silicones, dimers derived from a-alkylstyrene, 4,4-diarylbutadiene compounds, and mixtures thereof.

When present, the amount of UV filter in the composition may be from 0.01 to 30% by weight, preferably from 0.1 to 25% by weight, and more preferably from 5 to 20% by weight, relative to the total weight of the composition.

Mattifying Agents

A mattifying agent may also be included. Mattifying agents may include talc, silicas, silicone elastomers, polyamides, waxes, and combinations thereof. In addition to reducing the shine or appearance of oiliness, the mattifying agents can contribute to the overall texture and thickness of the cosmetic compositions. In some instances, one or more of the mattifying agents may be a particulate material or powder. Non-limiting examples of mattifying agents include methyl methacrylate/glycol dimethacrylate crosspolymer, vinyl dimethicone/methicone silsesquioxane crosspolymer, methyl methacrylate crosspolymer, nylon-12, polyamides, polyethylene, talc, titanium dioxide, silica, aluminum starch octenylsuccinate, or a mixture thereof.

Rheology Modifying Agents

In some embodiments, the copolymer is the only rheology modifying agent in the composition. In other embodiments, one or more additional rheology modifying agents may be present. The additional rheology modifying agents may be present in an amount from about 0.1 wt. % to about 20 wt. %.

The one or more rheology modifying agents may be xanthan gum, guar gum, biosaccharide gum, cellulose, acacia Seneca gum, sclerotium gum, agarose, pechtin, gellan gum, hyaluronic acid. Additionally, the one or more rheology modifying agents may include polymeric rheology modifying agents selected from the group consisting of ammonium polyacryloyldimethyl taurate, ammonium acryloyldimethyltaurate/VP copolymer, sodium polyacrylate, acrylates copolymers, polyacrylamide, carbomer, and acrylates/C10-30 alkyl acrylate crosspolymer. In some cases, the composition includes ammonium polyacryloyldimethyl taurate and/or sodium polyacrylate.

Many rheology modifying agents are water-soluble, and increase the viscosity of water or form an aqueous gel when the cosmetic composition of the invention is dispersed/dissolved in water. The aqueous solution may be heated and cooled, or neutralized, for forming the gel, if necessary. The rheology modifying agents may be dispersed/dissolved in an aqueous solvent that is soluble in water, e.g., ethyl alcohol when it is dispersed/dissolved in water. Non-limiting examples of various types of rheology modifying agents include:

a. Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include Ultrez® 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer rheology modifying agents useful herein are those selected from carbomers, acrylates/C10-C30 alkyl acrylate crosspolymers, and mixtures thereof.

b. Crosslinked Polyacrylate Polymers

The compositions of the present disclosure can optionally contain crosslinked polyacrylate polymers useful as rheology modifying agents or gelling agents including both cationic and nonionic polymers. Examples of useful crosslinked nonionic polyacrylate polymers and crosslinked cationic polyacrylate polymers are those described in U.S. Pat. Nos. 5,100,660, 4,849,484, 4,835,206, 4,628,078 4,599,379 and EP 228,868, which are all incorporated herein by reference in their entirety.

c. Polyacrylamide Polymers

The compositions of the present disclosure can optionally contain polyacrylamide polymers, especially nonionic polyacrylamide polymers including substituted branched or unbranched polymers. Among these polyacrylamide polymers is the nonionic polymer given the CTFA designation polyacrylamide and isoparaffin and laureth-7, available under the Tradename Sepigel 305 from Seppic Corporation.

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

The compositions may also contain thickening and texturising gels of the type as exemplified by the product range called Lubrajel® from United Guardian. These gels have moisturizing, viscosifying, stabilizing properties.

d. Polysaccharides

A wide variety of polysaccharides can be useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation.

Other useful polysaccharides include scleroglucans comprising a linear chain of (1-3) linked glucose units with a (1-6) linked glucose every three units, a commercially available example of which is Clearogel™. CS11 from Michel Mercier Products Inc.

e. Gums

Other thickening and gelling agents useful herein include materials which are primarily derived from natural sources. Nonlimiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Additional examples of water-soluble rheology modifying agents include water-soluble natural polymers, water-soluble synthetic polymers, clay minerals and silicic anhydride. Non-limiting examples of water-soluble natural polymers include gum arabic, tragacanth gum, karaya gum, guar gum, gellan gum, tara gum, locust bean gum, tamarind gum, sodium alginate, alginic acid propyleneglycol ester, carrageenan, farcelluran, agar, high-methoxy pectin, low-methoxy pectin, xanthine, chitosan, starch (for example starch derived from corn, potato, wheat, rice, sweet potato and tapioca, a-starch, soluble starch), fermentation polysaccharide (for example, xanthan gum, pullulan, carciran, dextran), acidic hetero-polysaccharide derived from callus of plants belonging to Polyantes sp. (for example, tuberous polysaccharide), proteins (for example, sodium casein, gelatin, albumin), chondroitin sulfate, and hyaluronic acid.

Non-limiting examples of water-soluble synthetic polymers include polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and starch derivatives (for example, starch oxide, dialdehyde starch, dextrin, British gum, acetyl starch, starch phosphate, carboxymethyl starch, hydroxyethyl starch, hydroxypropyl starch).

The composition may also contain a secondary film former.

As used herein, “secondary film-former” is meant to include one or more polymers having a combined concentration in the mascara composition that is less than the concentration of the “primary” (i.e., the vinylpyrrolidone copolymer described previously).

Non-limiting examples of secondary film formers include Polyimide-1, polyacrylic acid, polycrotonic acid, polymethacrylic acid, polymaleic acid, polyitaconic acid, octylacrylamide/acrylates/butylaminoethyl methacrylate copolymer, acrylates/octylacrylamide copolymer, acrylates copolymer, octylacrylamide/butylaminoethyl methacrylate copolymer, VA/crotonates/vinyl neodecanoate copolymer, VA/Crotonates copolymer, sodium polystyrene sulfonate, polyurethane-14 (and) AMP-Acrylates copolymer, acrylates/octylacrylarnide copolymer, acrylates/steareth-20 itaconate, copolymer, acrylates/ceteth-20 naconate copolymer, PVM/MA half ethyl ester copolymer, butylated PVP, PVP/hexadecene copolymer, PVP/eicosene copolymer, tricontanyl PVP, butyl ester of PVM/MA copolymer, PVM/MA copolymer, ethyl ester of PVM/MA copolymer, butyl ester of PVM/MA copolymer, vinyl caprolactam/PVP/dimethylaminoethyl methacrylate copolymer, polyquaternium-11, polyquaternium-28, PVPNinylcaprolactam/DMAPA Acrylates Copolymer, Isobutylene/Ethylmeleimide/Hydroxyethyimaleimide Copolymer, Acrylates/C1-2 Succinates/Hydroxyacrylates Copolymer, PVP/DMAPA Acrylates Copolymer, VP/Acrylates/Lauryl Methacrylate Copolymer, Pylyuaternium-55, PVP/Dimethylaminoethylmethacrylate Copolymer, VinylcaprolactamNP/Dimethylaminoethyl Methacrylate Copolymer, VA/Butyl Maleate/Isobornyl Acrylate Copolymer, acrylic acid/ethyl acrylate/t-butyl acrylamide, t-butyl acrylate/ethyl acrylate/methacrylic acid, ethyl acrylate/t-butyl acrylate/methacrylic acid, polyquaternium-16, polyquaternium-16, PVP, PVPNA copolymer, PVPNA copolymer, Polyurethane-1, VP/MethacrylamideNinyl Imidazole Copolymer, Acrylates Copolymer, Acrylates/Acrylamide Copolymer, Polyvinylcaprolactam, Dimethicone/Acrylates Copolymer, Amerchol, acrylic acid/methacrylic acid/acrylates/methacrylates, acrylic acid/methacrylic acid/acrylates/methacryates/hydroxy ester acrylates; methacryloyl ethyl betaine/acrylates copolymer, Acrylates/Hydroxyesters Acrylates Copolymer, Ethylenecarboxyamide/AMPSA/Methacrylates Copolymer, methacrylic acid/sodium acrylamidomethyl propane sulfonate copolymer, AMP-Acrylates/Allyl Methacrylate Copolymer, Polyacrylates-X, Acrylates/C10-30 Alkyl Acrylates Copolymer, Acrylates Copolymer, Polyurethane-2, Polyurethane-4, PPG-17/IPDI/DMPA Copolymer, polyethylene glycol; water-soluble acrylics; water-soluble polyesters; polyacrylamides, polyamines; polyquaternary amines; styrene maleic anhydride (SMA) resin; or mixtures or combinations thereof.

The composition may also contain a filler. Non-limiting examples of fillers include cellulose or cellulose derivatives, perlite, aerogel, talc, mica, silica, hollow silica microspheres, kaolin, calcium carbonate, magnesium carbonate, hydroxyapatite, boron nitride, glass or ceramic microcapsules, and composites of silica and of titanium dioxide, such as the TSG series sold by Nippon Sheet Glass, polyamide powders (Nylon® Orgasol from Atochem), polyethylene powder or polymethyl methacrylate powder, polytetrafluoroethylene (Teflon) powders, powders of acrylic acid copolymers (Polytrap from the company Dow Corning), lauroyllysine, hollow polymer microspheres such as those of polyvinylidene chloride/acrylonitrile, for instance Expancel (Nobel Industrie), hexamethylene diisocyanate/trimethylol hexyl lactone copolymer powder (Plastic Powder from Toshiki), silicone resin microbeads (for example Tospearl from Toshiba), natural or synthetic micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, Polypore® L 200 (Chemdal Corporation), and polyurethane powders, crosslinked polyurethane powders comprising a copolymer (e.g., a copolymer comprising trimethylol hexyl lactone), and a hexamethylene diisocyanate/trimethylol hexyl lactone polymer.

A second aspect of the present disclosure is a method for providing a composition with an improved wear resistance property, comprising the steps of first providing a copolymer of vinylpyrrolidone and a carboxylic acid monomer in water, then adding a neutralizing agent at an amount sufficient to neutralize less than 10% of the copolymer and/or adjust the pH of the water is less than about 5. A humectant may then be provided. As noted above, the humectant may be provided at any time—it may be provided first, and the copolymer neutralized either separately or in a mixture with the humectant, it may be provided simultaneously, or may be provided after the copolymer has been added, or after the copolymer has been neutralized.

Referring to FIG. 2, it can be seen that, sweat, and not water, impacts the rheology of a composition. A composition without humectant was used, and to that composition, varying levels of either sweat or additional water was added, then the rheological properties were measured. As can be seen in FIG. 2, the addition of sweat causes an increase in the complex shear modulus from about 200 Pa at 1% sweat (210) to about 588 Pa at 20% sweat (215). However, the addition of water causes a decrease in the complex shear modulus, from about 140 Pa at 1% additional water (220) to about 106 Pa at 20% additional water (225).

Thus, a third aspect of the present disclosure is a method for providing a long-lasting composition to the skin. The method includes first applying to a person's skin a composition comprising a copolymer of vinylpyrrolidone and a carboxylic acid monomer in water and a humectant, and then allowing the person to sweat, such that the sweat from the person increases the composition's overall resistance to deformation.

### EXAMPLE

An embodiment of a disclosed composition can be seen in Table 1, below.

TABLE 1

| Material | % w/w |
|---|---|
| Water | 50-95% |
| Copolymer | 1-10% |
| Neutralizing Agent | ≤2% |
| Humectant | ≤10% |
| Secondary Film Former | ≤10% |
| Filler | ≤4% |
| Emulsifier | ≤4% |
| Oil Phase | ≤40% |
| Pigments (Iron Oxides) | ≤15% |
| Preservatives | ≤2% |

The compositions were prepared as follows. The ingredients were weighed out, and the water, copolymer, and neutralizing agent were combined, then heated to 70° C. and mixed. Then, the humectant, secondary film former, filler, emulsifier, oil phase, and pigments were added and mixed. After cooling to below 40° C., the preservatives were added. The composition thus obtained was transferred into a closed jar to prevent it from drying out on contact with air; then allowed to sit for 24 hours before checking the homogeneity of the formulation.

In some embodiments, the shear viscosity of the compositions, when tested at 25° C. with a Brookfield viscometer, falls between 100 and 1200 Pa-s.

Evaluations

Three competitive foundation products were compared with the base formula with 4% neutralization (exemplary formula 1) and the base formula with 75% neutralization (comparative formula 2).

TABLE 2
Comparison of Exemplary Formula 1 and Comparative Formula 2

| | Exemplary | Comparative |
|---|---|---|
| | Formula 1 | Formula 1 |
| Material | % w/w | % w/w |
| Water | 80-99% | 80-99% |
| Copolymer | 1-10% | 1-10% |
| Neutralizing Agent | <1% | <2% |
| Humectant | <10% | <10% |
| Preservatives | <1% | <1% |

To measure transfer resistance of each formula, a 1 ml drawdown bar was used to drawdown test samples (primer) onto Bioskin (a polyurethane elastomer whose surface characteristics are similar to those of the skin) and then left to dry for 10 to 20 minutes. Afterwards, a 3 ml drawdown bar was used to drawdown the foundation on top of the original 1 ml drawdown samples and let it dry for overnight (8 hours). A pipette was then used to apply a drop of artificial sweat and artificial sebum on top of the drawdown samples and wait for 5 minutes. A clean Kimwipe™ disposable wipe was then used to rub the drawdown samples 5 times (each back-and-forth counting as 1 time) across where the artificial sweat and artificial sebum were applied. The transfer resistance was assessed by evaluating the amount of foundation that been transfer (rubbed) to the kimwipe. The lighter the color on the kimwipe the better the transfer resistance for the sample. Exemplary formula 1 performed substantially better than any other formula.

Those skilled in the art will recognize or be able to ascertain using no more than routine experimentation, many equivalents to the specific embodiments of the invention described herein. Such equivalents are intended to be encompassed by the following claims.

1. An aqueous leave-on composition, comprising:
a copolymer of vinylpyrrolidone and a carboxylic acid monomer in water; and
a humectant,
wherein the copolymer is present in an amount that is at least about 1% by weight of the total composition,
wherein the humectant is present in an amount that is less than about 10% by weight of the total composition, and
wherein the pH of the water is less than about 5, the copolymer is less than about 10% neutralized, or a combination thereof.

2. The aqueous leave-on composition according to claim 1, wherein the carboxylic acid monomer is acrylic acid.

3. The aqueous leave-on composition according to claim 1, wherein the pH of the aqueous phase is less than about 5 and the copolymer is less than about 10% neutralized.

4. The aqueous leave-on composition according to claim 1, wherein the pH of the aqueous phase is less than 4.

5. The aqueous leave-on composition according to claim 1, wherein the copolymer is less than 5% neutralized.

6. The aqueous leave-on composition according to claim 1, wherein the copolymer is present in an amount of between 1% and 10% by weight of the total composition.

7. The aqueous leave-on composition according to claim 1, wherein the humectant is present in an amount of between 0.1% and 6% by weight of the total composition.

8. The aqueous leave-on composition according to claim 1, wherein the humectant is a diol or triol.

9. The aqueous leave-on composition according to claim 8, wherein the humectant is butylene glycol.

10. The aqueous leave-on composition of claim 1, wherein the composition comprises at least two immiscible components prior to application.

11. The aqueous leave-on composition according to claim 1, further comprising a colorant.

12. The aqueous leave-on composition according to claim 11, wherein the colorant is an inorganic pigment.

13. The aqueous leave-on composition according to claim 1, further comprising a skin active agent, skin adjuvant, or UV filter.

14. The aqueous leave-on composition according to claim 1, further comprising a mattifying agent selected from the group consisting of a talc, silica, silicone elastomer, polyamide, wax, and combinations thereof.

15. The aqueous leave-on composition according to claim 1, wherein the copolymer is the only rheology modifying agent or film former in the composition.

16. The aqueous leave-on composition according to claim 1, where the complex shear modulus (G*) is between 100 and 1000 Pa.

17. The aqueous leave-on composition according to claim 1, where the shear viscosity at 25° C. is between 100 and 1200 Pa-s.

18. A method for providing a composition with an improved wear resistance property, comprising the steps of:
providing a copolymer of vinylpyrrolidone and a carboxylic acid monomer in water; and
adding a neutralizing agent at an amount sufficient to neutralize less than 10% of the copolymer, adjust the pH of the water is less than about 5, or a combination thereof; and
providing a humectant,
wherein the copolymer is present in an amount that is at least about 1% by weight of the total composition, and
wherein the humectant is present in an amount that is at least about 10% by weight of the total composition.

19. The method according to claim 18, wherein the neutralizing agent comprises amines.

20. A method for providing a long-lasting composition to the skin, comprising the steps of:
applying to a person's skin a composition comprising a copolymer of vinylpyrrolidone and a carboxylic acid monomer in water and a humectant; and
allowing the person to sweat,
wherein the composition is configured such that the sweat from the person increases the composition's overall resistance to deformation.
